(19) United States
(12) Patent Application Publication
(10) Pub. No.: US 2020/0060552 A1
(43) Pub. Date: Feb. 27, 2020

(54) BODY SURFACE TEMPERATURE MEASUREMENT DEVICE

(71) Applicant: Sharp Kabushiki Kaisha, Sakai City, Osaka (JP)

(72) Inventors: Azusa NAKANO, Sakai City (JP); Tetsuya HAYASHI, Sakai City (JP); Hiroshi SAKAYA, Sakai City (JP)

(21) Appl. No.: 16/466,085

(22) PCT Filed: Nov. 30, 2017
(86) PCT No.: PCT/JP2017/042949

§ 371 Date: Jun. 3, 2019

(30) Foreign Application Priority Data
Dec. 5, 2016 (JP) 2016-235703

(51) Int. Cl. A61B 5/01; G01J 5/02; G01J 5/04; G01J 5/00; A61B 5/00; A61D 13/00

(52) U.S. Cl. CPC A61B 5/01; G01J 5/021; G01J 5/04; G01J 5/0025; A61B 2503/40; A61D 13/00; A61B 2562/0271; A61B 2560/0406; A61B 5/6831

(57) ABSTRACT

A body surface temperature measurement device capable of appropriately performing measurement of a body surface temperature of an animal is realized. A body surface temperature measurement device includes a temperature sensing unit including an infrared ray detection sensor and a cover that covers the infrared ray detection sensor, in which the cover closely contacts a body surface of the animal and at least a part of a contact surface of the cover, which contacts the body surface of the animal, is formed into a curved surface that bulges toward a body surface side of the animal.

### TECHNICAL FIELD

The present disclosure relates to a body surface temperature measurement device that measures a body surface temperature of an animal.

### BACKGROUND ART

In recent years, it has been recognized by an owner that daily health care is important for an animal such as a companion animal (pet). In order to grasp a health condition of the animal, it is important to measure a body temperature of the animal. In measurement of the body temperature of the animal, for example, in a case of a dog, a probe thermometer that includes a thermistor is inserted into a rectum to measure a rectal temperature. However, in a normal home, the measurement of the rectal temperature of the animal is a difficult work.

On the other hand, as a thermometer other than the probe thermometer, an infrared radiation thermometer or the like is also used in measurement of a body temperature of an animal including a human besides the companion animal. For example, PTL 1 discloses an infrared thermometer that is able to measure an accurate body temperature regardless of a distance by using an infrared sensor. Specifically, the infrared thermometer measures an electrostatic capacity when a proximity sensor constituted by a ground electrode and an electrode comes close to a human body. The infrared thermometer compares a predetermined electrostatic capacity to the electrostatic capacity that is measured with the proximity sensor. When the measured electrostatic capacity reaches the predetermined electrostatic capacity, the infrared thermometer determines that a main body of the sensor contacts the human body. Then, the infrared thermometer calculates a body temperature based on an amount of infrared radiation from the infrared sensor at this time.

### CITATION LIST

### Patent Literature

PTL 1: Japanese Unexamined Patent Application Publication No. 2012-217563 (published on Nov. 12, 2012)

### SUMMARY OF INVENTION

### Technical Problem

In the related art as described above, however, in a case of measuring a body temperature of an animal, it is difficult to measure the body temperature appropriately due to the following factors or the like.

Factor 1: A situation in which a body temperature is measured is a particular situation for an animal. Accordingly, the animal gets excited (for example, behaves violently) when the body temperature is intended to be measured. Thus, it is difficult to measure the body temperature of the animal at a rest state appropriately.

Factor 2: Also in a case where a body surface temperature (temperature near a body surface) is measured instead of measuring the body temperature, body hair becomes an obstacle. Thus, ingenuity is required to measure the body surface temperature of the animal appropriately.

### Solution to Problem

In order to solve the aforementioned problems, a body surface temperature measurement device is a body surface temperature measurement device that measures a body surface temperature of an animal, and includes: a temperature sensing unit that includes an infrared ray detection sensor and a cover that covers the infrared ray detection sensor; and a belt that causes the temperature sensing unit to closely contact a body surface of the animal or that fixes the temperature sensing unit to a collar to be mounted on the animal or a body belt to be mounted on the animal; in which in a case where the body surface temperature measurement device is mounted on the animal, the cover is pressed against the body surface of the animal, and at least a part of a contact surface of the cover, which contacts the body surface of the animal, is formed into a curved surface that bulges toward a body surface side of the animal.

### Advantageous Effects of Invention

An effect that appropriate body surface measurement is able to be performed in measurement of a body surface of an animal is exerted.

### BRIEF DESCRIPTION OF DRAWINGS

FIGS. 1(a) and 1(b) are views each illustrating an example of a structure of a body surface temperature measurement device.

FIG. 2 is a view illustrating another example of a configuration of a main part of a measurement temperature calculation apparatus.

FIG. 3 is a view illustrating an example of a configuration of hardware of the measurement temperature calculation apparatus.

FIG. 4 is a view illustrating a region in which a temperature sensing unit is preferably arranged.

FIG. 5 is a view illustrating a state in which the body surface temperature measurement device is mounted on a dog.

FIG. 6(a) is a view illustrating a state in which the body surface temperature measurement device is appropriately mounted on the dog and FIG. 6(b) is a view illustrating a state in which the body surface temperature measurement device is mounted on the dog while the temperature sensing unit is inclined.

FIG. 7(a) illustrates distribution obtained by plotting a measurement value of a body surface temperature and a measurement value of a rectal temperature in association with each other, FIG. 7(b) indicates an average value of differences between the measurement value (with a lens or without a lens) of the body surface temperature and the measurement value of the rectal temperature, and FIG. 7(c) indicates standard deviation of a difference between the measurement value (with a lens or without a lens) of the body surface temperature and the measurement value of the rectal temperature.

FIG. 8(a) is a view illustrating an example of a structure of the temperature sensing unit, FIG. 8(b) is a view illustrating a state in which the body surface temperature measurement device is appropriately mounted on the dog, FIG. 8(c) is a view illustrating a state in which the body surface temperature measurement device is mounted on the dog while the temperature sensing unit is inclined, FIG. 8(d) is a view illustrating a state in which the body surface temperature measurement device is mounted on the dog while the temperature sensing unit is inclined, and FIG. 8(e) is a view illustrating a state in which the body surface temperature measurement device is mounted on the dog while the temperature sensing unit is more inclined compared to the state illustrated in FIG. 8(c).

FIGS. 9(a) and 9(b) are views each illustrating an example of a structure of a body surface temperature measurement device.

FIG. 10(a) is a view illustrating a position of mounting the body surface temperature measurement device, FIG. 10(b) is a view illustrating a state in which the body surface temperature measurement device is mounted on the dog, and FIGS. 10(c) to 10(e) are views each illustrating a position of mounting another body surface temperature measurement device.

FIGS. 11(a) and 11(b) are views each illustrating an example of a structure of a body surface temperature measurement device.

FIGS. 12(a) and 12(b) are views each illustrating an example of a structure of an adjuster.

FIGS. 13(a) and 13(b) are views each illustrating an example of a structure of a body surface temperature measurement device.

FIG. 14 is a view illustrating an example of a configuration of a main part of a measurement temperature calculation apparatus.

### DESCRIPTION OF EMBODIMENTS

Hereinafter, an example in which a dog is a target of a body surface temperature measurement device according to an embodiment will be described. An animal to be a target of body surface temperature measurement is not particularly limited to a dog, and for example, may be a companion animal such as a cat, a rabbit, a ferret, a monkey, or a hamster, livestock such as a horse, cattle, a pig, a sheep, or a goat, or an animal such as a tiger or a lion that is bred in a zoo.

### Embodiment 1

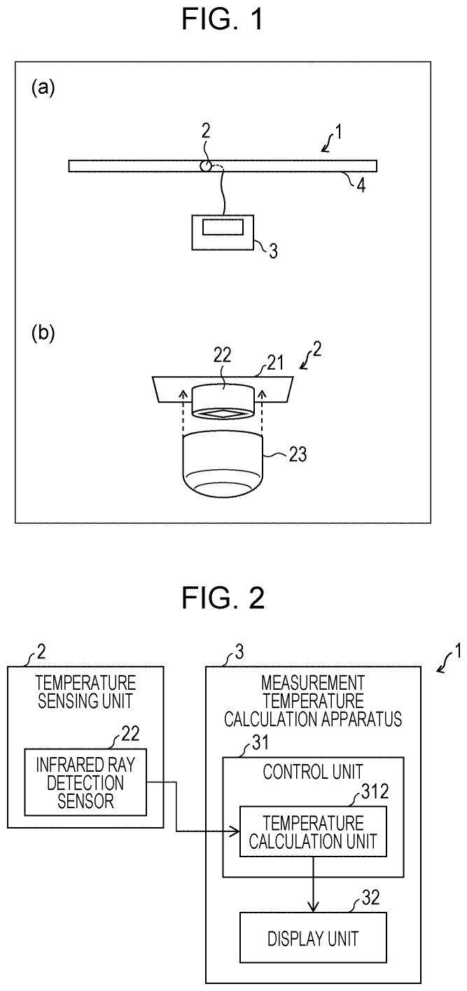

An embodiment will be described below in detail with reference to FIGS. 1 to 7.

First, a structure of a body surface temperature measurement device 1 will be described with use of FIG. 1. FIG. 1 is a view illustrating an example of the structure of the body surface temperature measurement device 1. FIG. 1(a) is an external view of the body surface temperature measurement device 1. As illustrated in FIG. 1(a), the body surface temperature measurement device 1 includes a temperature sensing unit 2, a measurement temperature calculation apparatus 3, and a mounting belt (belt) 4.

The temperature sensing unit 2 detects a body surface temperature of an animal. FIG. 1(b) is a view illustrating a structure of the temperature sensing unit 2. As illustrated in FIG. 1(b), the temperature sensing unit 2 includes a substrate 21, an infrared ray detection sensor 22, and a cover 23. The infrared ray detection sensor 22 is arranged on the substrate 21. The infrared ray detection sensor 22 is only required to be a sensor capable of detecting an infrared ray, and is able to use a thermopile as an example. As illustrated in FIG. 1(b), the cover 23 is arranged so as to cover the infrared ray detection sensor 22. At a time of measuring a body surface temperature of an animal, the cover 23 is pressed so as to closely contact a body surface of the animal. At least a part of a contact surface of the cover 23, which contacts the body surface of the animal, is formed to have a convex surface that bulges toward a body surface side of the animal. The convex surface may be configured by combining a plurality of polygonal planes such as a surface obtained by performing diamond cut, or may be configured by a round and smooth curved surface (such a convex surface is merely described as a “curved surface” in the present specification). Specifically, the curved surface of the cover 23 is formed so that a distance between (i) any contact point of the body surface of the animal and the cover 23 and (ii) a center of the infrared ray detection sensor 22 is substantially constant (for example, 2 mm). For example, the curved surface has a shape of a part of a spherical surface (for example, a sphere having approximately 1 cm diameter). It is preferable that the cover 23 is made from a material such as polyethylene, which has characteristics of hardly absorbing an infrared ray. The cover 23 may also be configured to include a condensing lens. The contact surface of the cover 23, which contacts the body surface of the animal, may function as a light receiving surface of the condensing lens accommodated in the cover 23. The condensing lens is not particularly limited and may be a Fresnel lens as an example.

The measurement temperature calculation apparatus 3 calculates a measurement temperature from a detection value that is detected by the temperature sensing unit 2. The measurement temperature calculation apparatus 3 may include a display unit for displaying the measurement temperature, an operation status, or the like. The measurement temperature calculation apparatus 3 may also include an operation input unit for receiving an operation to start an operation of the sensor, an operation to end the operation of the sensor, a restarting operation, or the like. For example, the measurement temperature calculation apparatus 3 may be configured to perform an input of the operation described above by pressing a button of the operation input unit by a user. The measurement temperature calculation apparatus 3 is also able to be configured by including a microcomputer, a dedicated circuit, or the like for calculating the measurement temperature.

FIG. 2 is a view illustrating an example of a configuration of the measurement temperature calculation apparatus 3. As illustrated in FIG. 2, the measurement temperature calculation apparatus 3 includes a control unit 31 and a display unit 32. The control unit 31 includes a temperature calculation unit 312. The temperature calculation unit 312 calculates a measurement temperature from the detection value that is detected by the temperature sensing unit 2. The temperature calculation unit 312 transmits the calculated measurement temperature to the display unit 32. The display unit 32 displays the measurement temperature, an operation status of the measurement temperature calculation apparatus 3, or the like.

A control block (in particular, a detection value selection unit 311 and temperature calculation units 312 and 312a) of each of the measurement temperature calculation apparatus 3 and a measurement temperature calculation apparatus 3a that is described below may be realized by a logic circuit (hardware) formed in an integrated circuit (IC chip) or the like or may be realized by software with use of a CPU (Central Processing Unit).

In the latter case, each of the measurement temperature calculation apparatuses 3 and 3a includes a CPU that executes a command of a program that is software realizing each of functions, a ROM (Read Only Memory) or a storage apparatus (each referred to as a “recording medium”) in which the program and various kinds of data are stored so as to be readable by a computer (or a CPU), a RAM (Random Access Memory) that develops the program, and the like. An object of the disclosure is achieved by a computer (or a CPU) reading and executing the program from the recording medium. As the recording medium, a “non-transitory tangible medium”, for example, such as a tape, a disk, a card, a semiconductor memory, or a programmable logic circuit may be used. The program may be supplied to the computer via any transmission medium (such as a communication network or a broadcast wave) which enables the program to be transmitted. Note that, the disclosure can also be achieved even with a data signal actualized by electronic transmission of the above-described program and embedded in a carrier wave. A recording unit for recording a calculation result may be further provided.

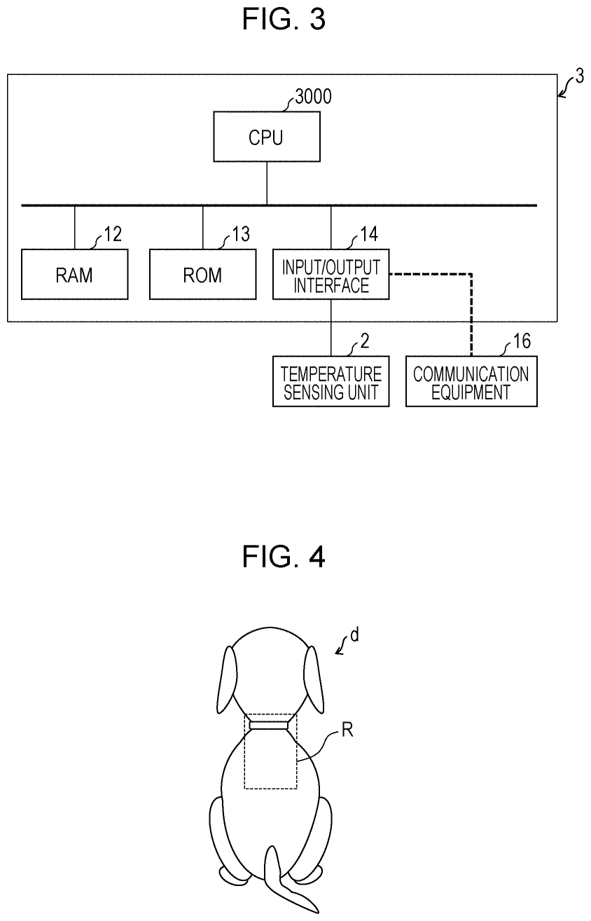

Moreover, by further providing a circuit for communication (communication in a wired or wireless manner) in the measurement temperature calculation apparatus 3 or 3a, it becomes possible to transmit a measurement (calculation) result to a different communication equipment 16 (external communication equipment, refer to FIG. 3). In this case, it also becomes possible to perform further comparison with another measurement value by using the received measurement result, accumulate the measurement result, or notify a user such as an owner of the measurement result. Examples of the different communication equipment include a smartphone, a portable phone, a tablet terminal, a computer (such as a PC), or a smart watch.

Here, an example of a configuration of hardware of the measurement temperature calculation apparatus 3 will be described with use of FIG. 3. FIG. 3 is a view illustrating an example of the configuration of the hardware of the measurement temperature calculation apparatus 3. The measurement temperature calculation apparatus 3 includes a CPU 3000, a RAM 12, and a ROM 13. An input/output interface 14 is connected to a bus that connects the CPU 3000, the ROM 13, and the RAM 12. The input/output interface 14 is an interface for performing, under control of the CPU 3000, data transmission and reception processing with various kinds of input/output equipment (for example, the temperature sensing unit 2, the external communication equipment 16, and the like) that are connected to the measurement temperature calculation apparatus 3. Note that, a broken line connecting the input/output interface 14 and the communication equipment 16, which is illustrated in FIG. 3, indicates communication in a wired or wireless manner.

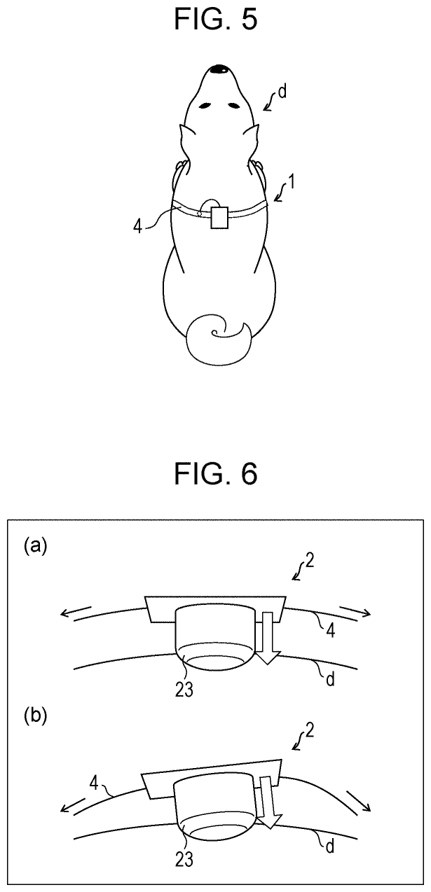

Description will be given with reference back to FIG. 1 again. The mounting belt 4 is pressed so as to make the temperature sensing unit 2 closely contact the body surface of the animal. The mounting belt 4 is a belt for mounting the body surface temperature measurement device 1 on the animal. More specifically, the mounting belt 4 applies tension to a circumference of a torso or a neck of the animal to cause the body surface temperature measurement device 1 to closely contact a body of the animal (refer to FIG. 5). The mounting belt 4 may be configured to include a buckle (fastener) at both ends. The mounting belt 4 is bonded to a surface of the substrate 21, which is on a back side of a surface of the substrate 21 where the infrared ray detection sensor 22 is arranged, or the mounting belt 4 includes a member by which the mounting belt 4 is fixed to the substrate 21. The mounting belt 4 is pressed so that the surface where the infrared ray detection sensor 22 of the temperature sensing unit 2 is arranged closely contacts the body surface of the animal in an opposing manner.

The measurement temperature calculation apparatus 3 may be configured to be combined with the mounting belt 4, for example. For example, the measurement temperature calculation apparatus 3 may be formed to be put on the back of the animal by mounting the mounting belt 4 on the animal. Moreover, the measurement temperature calculation apparatus 3 may be put into a container having a shape of a rucksack or the like and the container may be put on the back of the animal, or the measurement temperature calculation apparatus 3 may be put into a container having a shape of a pouch, a small article case, or the like and the container may be dangled from a collar. In a case of being sufficiently small, the measurement temperature calculation apparatus 3 may be secured to the collar or a body belt by using a fixing jig such as a clip. A part or all of the collar or the body belt that is mounted on the animal may be configured to have a cylindrical shape, and the measurement temperature calculation apparatus 3 may be inserted into the cylinder.

A mounting example of the body surface temperature measurement device 1 will be described with reference with FIGS. 4 and 5. FIG. 4 is a view illustrating an example of arrangement of temperature sensing unit 2. Here, an example of arrangement of the temperature sensing unit 2 on an animal to be measured is indicated. In the example illustrated in FIG. 4, a region R is a region in which the temperature sensing unit 2 is preferably arranged on a dog d. The region R is a region below a neck on a back side of the dog d and has the following features.

A forefoot or a hind foot of the dog d does not reach or is hard to reach. The temperature sensing unit 2 is not pinched between the animal and the ground (or a floor surface or the like) unless the dog d lies face up. A stain due to mud splash or the like hardly occurs, for example, while walking the dog d. Further, the region in which the temperature sensing unit 2 is preferably arranged is a region in which the temperature sensing unit 2 is less likely to be interfered, in other words, a region which is hardly affected by a movement of a bone caused by, for example, walking or changing a posture, and more specifically, a part that excludes a part around a spine and a part around a scapula and that is substantially flat.

Subsequently, the mounting example of the body surface temperature measurement device 1 will be described with reference to FIG. 5. FIG. 5 is a view illustrating a state in which the body surface temperature measurement device 1 is mounted on the dog d. In the example illustrated in FIG. 5, a configuration in which the body surface temperature measurement device 1 serves as a body belt is provided. The mounting belt 4 applies tension to a circumference of a torso of the animal, so that the temperature sensing unit 2 is caused to closely contact the body surface of the animal. As another mounting example, a configuration in which the body surface temperature measurement device 1 serves as a collar may be provided. In this configuration, the mounting belt 4 applies tension to a circumference of a neck of the target animal, so that the temperature sensing unit 2 is caused to closely contact the body of the animal.

Next, a state of the temperature sensing unit 2 when the body surface temperature measurement device 1 is mounted on the dog d will be described with reference to FIG. 6. FIG. 6(a) is a view illustrating a state in which the body surface temperature measurement device 1 is appropriately mounted on the dog d. FIG. 6(b) is a view illustrating a state in which the body surface temperature measurement device 1 is mounted on the dog d while the temperature sensing unit 2 is inclined. As illustrated in FIGS. 6(a) and 6(b), the cover 23 of the temperature sensing unit 2 is pressed against the body surface of the dog d and fixed by the tension of the mounting belt 4.

As illustrated in FIG. 6(a), in the state in which the temperature sensing unit 2 is appropriately fitted, (i) a contact surface of the cover 23 and the body surface of the dog d and (ii) the substrate 21 are parallel. On the other hand, as illustrated in FIG. 6(b), in the state in which the temperature sensing unit 2 is inclined, (i) the contact surface of the cover 23 and the body surface of the dog d and (ii) the substrate 21 are not parallel. The state illustrated in FIG. 6(b) is a state that occurs when the tension applied to both ends of the mounting belt 4 with respect to the substrate 21 is temporarily biased, but is immediately returned to the original state (FIG. 6(a)) in many cases. The state illustrated in FIG. 6(b) is kept in the following cases. For example, a case where the dog d lies down with one side of the mounting belt 4 being down while the tension of the both ends of the mounting belt 4 is biased and a case where the dog d leans against a wall at a corner of a room or the like so that a skin or flesh is concentrated to thereby incline the temperature sensing unit 2, the cover 23, and the substrate 21 are cited.

For example, a configuration in which the mounting belt 4 is connected to each of facing edges of the substrate 21 may be provided. In accordance with the configuration, even in a case where the temperature sensing unit 2 is temporarily inclined, the inclination of the temperature sensing unit 2 is able to be corrected by the tension of the mounting belt 4 and the temperature sensing unit 2 is able to be automatically returned to an appropriate state. Additionally, even in a case where the inclination of the temperature sensing unit 2 is not automatically returned to the appropriate state, when a user only corrects finely a degree of application of the tension of the mounting belt 4, the inclination of the temperature sensing unit 2 is able to be corrected so as to be an appropriate position (direction). As described above, the measurement temperature calculation apparatus 3 may be put into a container, the temperature sensing unit 2 may be connected to the container so as to face the body surface, and the mounting belt may be connected to both ends of the container. That is, the tension generated by the mounting belt 4 may act directly or indirectly on the temperature sensing unit 2 so that the temperature sensing unit 2 faces the body surface of the animal and closely contacts the body of the animal.

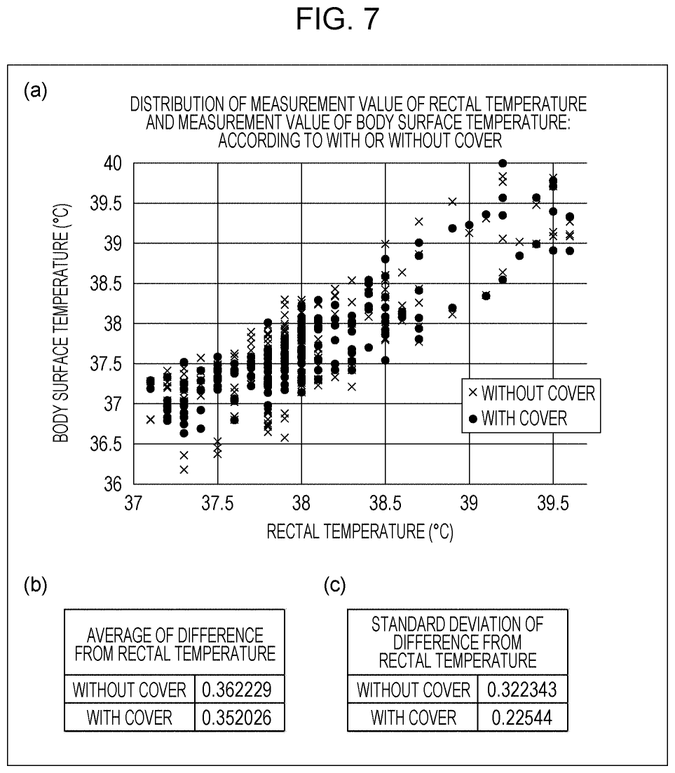

Next, an effect of the cover 23 will be described with reference to FIG. 7. FIG. 7(a) illustrates distribution obtained by plotting a measurement value of a rectal temperature and a measurement value of a body surface temperature in association with each other. A black dot (●) mark illustrated in FIG. 7(a) is a plot in which a measurement value (with the cover) of a body surface temperature that is measured by using the temperature sensing unit 2 that includes the cover 23 is associated with a measurement value of a rectal temperature. A cross (x) mark illustrated in FIG. 7(a) is a plot in which a measurement value (without the cover) of a body surface temperature that is measured by using a temperature sensing unit that does not include the cover 23 is associated with a measurement value of a rectal temperature. Note that, the cover 23 used in the measurement of the body surface temperature has the contact surface, which contacts the body surface of the animal, formed into a curved surface.

A vertical axis of a graph illustrated in FIG. 7(a) indicates the measurement value of the body surface temperature and a horizontal axis of the graph illustrated in FIG. 7(a) indicates the measurement value of the rectal temperature. As illustrated in FIG. 7(a), a value of the measurement with the cover varies little compared to a value of the measurement without the cover.

FIG. 7(b) indicates an average value of differences between the measurement value (with the cover or without the cover) of the body surface temperature and the measurement value of the rectal temperature. As illustrated in FIG. 7(b), the average value of the differences between the measurement value (with the cover) of the body surface temperature and the measurement value of the rectal temperature is 0.35226, and the average value of the differences between the measurement value (without the cover) of the body surface temperature and the measurement value of the rectal temperature is 0.362229. FIG. 7(c) indicates standard deviation of a difference between the measurement value (with the cover or without the cover) of the body surface temperature and the measurement value of the rectal temperature. The standard deviation of the difference between the measurement value (with the cover) of the body surface temperature and the measurement value of the rectal temperature is 0.22544, and the standard deviation of the difference between the measurement value (without the cover) of the body surface temperature and the measurement value of the rectal temperature is 0.322343. That is, a result indicating that measurement value (with the cover) of the body surface temperature has a smaller difference from the measurement value of the rectal temperature and has a suppressed variation compared to the measurement value (without the cover) of the body surface temperature is obtained. This result is obtained when the contact surface of the cover 23, which contacts the body surface of the animal, is formed into the curved surface and a distance between a light receiving unit of the infrared ray detection sensor 22 and the body surface of the animal is kept substantially constant, as described above. That is, it is easier to keep the distance between the light receiving unit of the infrared ray detection sensor 22 and the body surface of the animal fixed in the case where the contact surface of the cover 23 with the body surface is formed into the curved surface compared to a case where the contact surface of the cover 23 with the body surface is formed into a flat surface.

Note that, the curved surface of the cover 23 may be configured to function also as a condensing lens (for example, a Fresnel lens) to cause light to be more efficiently converged on the infrared ray detection sensor 22 while the distance between the light receiving unit of the infrared ray detection sensor 22 and the body surface of the animal is kept substantially constant. The Fresnel lens may be formed by forming, on an inside or an outside of the curved surface of the cover 23, a groove for converging light, for example.

According to the aforementioned configuration, the contact surface of the cover is pressed against the body surface of the animal. Thus, the body surface temperature measurement device is able to be mounted on an animal in a wearable manner and the body surface temperature measurement device is able to keep a state always allowing measurement of the body surface temperature. Therefore, the body surface temperature is able to be measured when the animal mounting the body surface temperature measurement device is in a rest state. Accordingly, the body surface temperature of the animal at rest is able to be detected. According to the aforementioned configuration, the contact surface of the cover, which contacts the body surface of the animal, is formed into the curved surface that bulges toward the body surface side of the animal. This makes it easy to keep a distance between a center of the infrared ray detection sensor and a measurement target (body surface) the same. It is possible to more accurately measure a body surface temperature of a hairy animal, compared to a configuration in which a housing mechanism by which a distance between the infrared ray detection sensor and the measurement target (such as an eardrum or skin) is kept constant is provided. Additionally, the cover makes it possible to reduce stain on the infrared ray detection sensor.

According to the aforementioned body surface temperature measurement device, the body surface temperature of the animal is detected by using the infrared ray detection sensor. Thus, a body surface temperature of an animal that has difficulty in keeping a rest state is also able to be detected stably compared to a configuration in which the body surface temperature is detected by using a thermistor that sensitively reacts a change of an external environment. Accordingly, in measurement of a body surface temperature of an animal, the body surface temperature measurement device is able to perform appropriate body surface temperature measurement.

Furthermore, in a configuration in which the contact surface of the cover functions also as a light condensing surface of the condensing lens, even when an angle formed by the infrared ray detection sensor and the body surface of the animal is changed, the condensing lens condenses an infrared ray on the infrared ray detection sensor, so that influence on the detection of the body surface temperature due to the change is able to be reduced.

Note that, since it is well known that the thermistor is often used together with the infrared ray detection sensor in order to measure a temperature, detailed description will not be given here.

### Embodiment 2

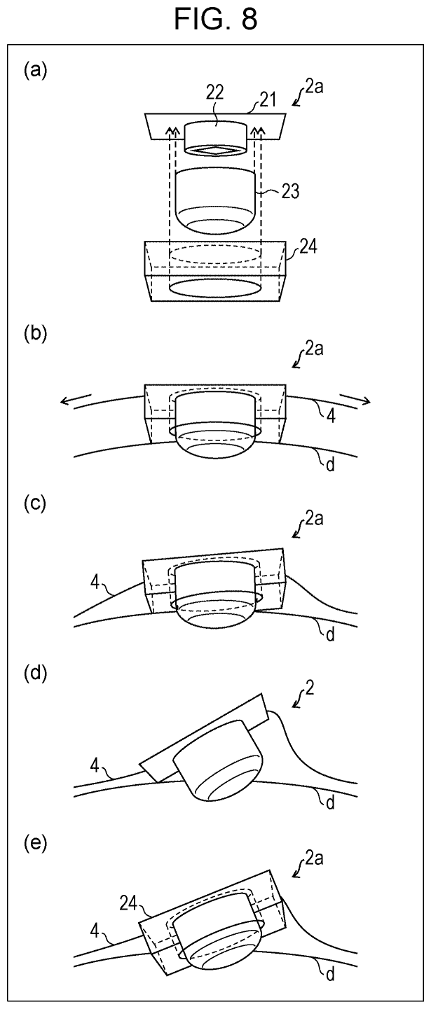

Another embodiment will be described as follows with reference to FIG. 8. Note that, for convenience of description, a member having the same function as that of the member described in the aforementioned embodiment will be given the same reference sign and description thereof will be omitted.

A temperature sensing unit 2a according to the present embodiment includes an auxiliary pedestal 24.

A structure of the temperature sensing unit 2a will be described with reference to FIG. 8. FIG. 8(a) is a view illustrating the structure of the temperature sensing unit 2a. FIG. 8(b) is a view illustrating a state in which the body surface temperature measurement device 1 is appropriately mounted on the dog d. As illustrated in FIGS. 8(a) and 8(b), the auxiliary pedestal 24 covers at least a part of the cover 23. In a case where the temperature sensing unit 2a is inclined against the body surface of the animal, the auxiliary pedestal 24 contacts the body surface to suppress the inclination of the temperature sensing unit 2a. In detail, a hole is provided in the auxiliary pedestal 24. The hole is formed so that the cover 23 is fit therein. In a state in which the cover 23 is inserted and fit into the hole provided in the auxiliary pedestal 24, an infrared ray transmitting surface (light condensing surface) of the cover 23 is exposed from the hole. Thus, the cover 23 is pressed against the body surface of the animal by the mounting belt 4. Moreover, the auxiliary pedestal 24 may be made from an elastic member.

An effect of the auxiliary pedestal 24 will be described with reference to FIGS. 8(c) to 8(e). FIG. 8(c) is a view illustrating a state in which the body surface temperature measurement device 1 is mounted on the dog d while the temperature sensing unit 2a is inclined.

As illustrated in FIG. 8(c), in the temperature sensing unit 2a, it is possible to suppress inclination of the temperature sensing unit 2a, which is caused when any one side of the mounting belt 4 is pulled. Furthermore, when the auxiliary pedestal 24 is made from an elastic member, the auxiliary pedestal 24 absorbs the inclination of the temperature sensing unit 2a.

FIG. 8(d) is a view illustrating a state in which the body surface temperature measurement device 1 is mounted on the dog d while the temperature sensing unit 2 described in Embodiment 1 is inclined. The inclination of the temperature sensing unit 2 is larger than the inclination of the temperature sensing unit 2a which is illustrated in FIG. 8(c). There is a possibility that the light condensing surface of the cover 23 is not able to contact the body surface of the animal because a force is further applied to one side of the mounting belt 4. There is also a possibility that a corner of the cover 23 or an edge of the substrate 21 contacts the body surface of the animal. FIG. 8(e) is a view illustrating a state in which the body surface temperature measurement device 1 is mounted on the dog d while the temperature sensing unit 2a is inclined compared to the state illustrated in FIG. 8(c). As illustrated in FIG. 8(e), even in a case where the temperature sensing unit 2 or the temperature sensing unit 2a is greatly inclined, a cushioning effect is exerted by the auxiliary pedestal 24 in the temperature sensing unit 2a that includes the auxiliary pedestal 24 made from the elastic member. Thus, a degree of encroaching on the body surface of the animal by the substrate 21 or the cover 23 is reduced.

The auxiliary pedestal 24 may be made from a material having high friction with body hair of the body surface of the animal. According to such a configuration, it is possible to suppress deviation between the temperature sensing unit 2a and the body surface of the animal. The auxiliary pedestal 24 may be made from a material having low friction with the body hair of the body surface of the animal. According to such a configuration, even in a case where a position of the temperature sensing unit 2a is deviated, the temperature sensing unit 2a is less likely to pull the hair of the body surface of the animal. Thus, the animal mounting the body surface temperature measurement device 1 is able to obtain a comfortable wearing feeling.

Examples of a material or a member that constitutes the auxiliary pedestal 24 include rubber, urethane, silicone, cloth (natural fiber such as cotton or silk, chemical fiber such as nylon, polyester, or rayon, or the like) into which formable beads or inner cotton is packed to form a cushion shape. Note that, in a case where a material that is not an elastic body, such as metal or wood, is used, corners of the auxiliary pedestal 24 are preferably cut off so that the animal does not feel pain when the corners hit the body surface of the animal.

According to the aforementioned configuration, it is possible to suppress the inclination of the temperature sensing unit against the body surface by providing the auxiliary pedestal in the temperature sensing unit. Moreover, according to the aforementioned configuration, a degree of encroaching on the body surface of the animal by the temperature sensing unit is reduced.

Furthermore, in the body surface temperature measurement device of the present embodiment, the auxiliary pedestal may be made from the elastic member. According to the configuration described above, the auxiliary pedestal more easily absorbs inclination of the sensor.

### Embodiment 3

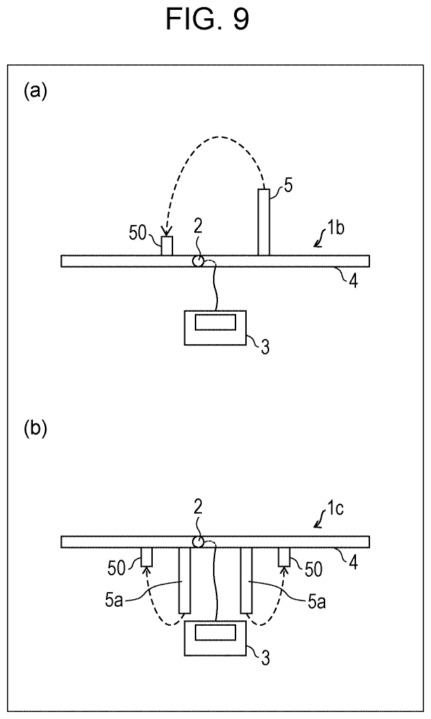

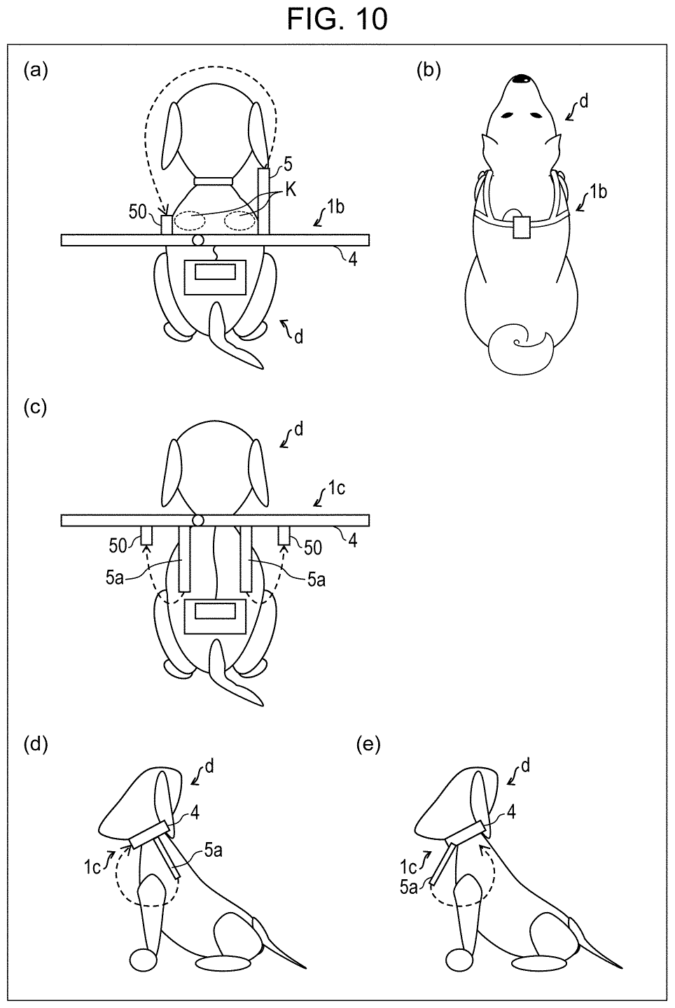

Another embodiment of the disclosure will be described as follows with reference to FIGS. 9 and 10. Note that, for convenience of description, a member having the same function as that of the member described in the aforementioned embodiments will be given the same reference sign and description thereof will be omitted. A body surface temperature measurement device 1b and a body surface temperature measurement device 1c according to the present embodiment respectively include an auxiliary belt 5 and an auxiliary belt 5a each of which fixes a position of the mounting belt 4 on the body surface of the animal.

A structure of the body surface temperature measurement device 1b in which the mounting belt 4 has a shape of a body belt will be described with reference to FIGS. 9 and 10. FIG. 9(a) is a view illustrating the structure of the body surface temperature measurement device 1b. As illustrated in FIG. 9(a), the mounting belt 4 includes the auxiliary belt 5 and a buckle 50.

FIG. 10(a) is a view illustrating a position of mounting the body surface temperature measurement device 1b and FIG. 10(b) is a view illustrating a state in which the body surface temperature measurement device 1b is mounted on the dog d. As illustrated in FIGS. 10(a) and 10(b), in a case where the body surface temperature measurement device 1b is mounted on the dog d, the mounting belt 4 contacts a circumference of a torso that is closer to a tail of the dog d than right and left forefeet thereof and includes the auxiliary belt 5 that makes contact across a neck on a ventral side of the dog d. For detailed explanation, as illustrated in FIG. 10(a), the mounting belt 4 applies tension to the circumference of the torso closer to the tail of the dog d than a scapula K thereof so that the body surface temperature measurement device 1b closely contacts the body of the dog d. In the state in which the body surface temperature measurement device 1b is mounted, the auxiliary belt 5 connected to the mounting belt 4 makes contact across the ventral side of the dock d from a back side thereof. Moreover, the auxiliary belt 5 includes a fastener at a tip to be connected to the buckle 50.

According to the aforementioned configuration, the auxiliary belt 5 suppresses rotation of the mounting belt 4 having the shape of the body belt. Thus, the auxiliary belt 5 suppresses a case where the temperature sensing unit 2 moves to a region in which a foot of the dog d reaches or a case where the temperature sensing unit 2 moves to a region in which the temperature sensing unit 2 is easily buried by something. The auxiliary belt 5 is able to prevent the mounting belt 4 having the shape of the body belt from moving toward a side (hind foot direction) of a thinner body of the dog d.

Next, a structure of the body surface temperature measurement device 1c in which the mounting belt 4 has a shape of a collar will be described with reference to FIGS. 9 and 10. FIG. 9(b) is a view illustrating the structure of the body surface temperature measurement device 1c. As illustrated in FIG. 9(b), the mounting belt 4 includes auxiliary belts 5a and buckles 50.

FIGS. 10(c) to 10(e) are views each illustrating a position of mounting the body surface temperature measurement device 1c. As illustrated in FIGS. 10(c) to 10(e), in a case where the body surface temperature measurement device 1c is mounted on the dog d, the mounting belt 4 contacts a circumference of the neck of the dog d. Moreover, in a case where the body surface temperature measurement device 1c is mounted on the dog d, the auxiliary belts 5a contact right and left axillae of the dog d. For detailed explanation, as illustrated in FIGS. 10(c) and 10(d), the mounting belt 4 applies tension to the circumference of the neck of the dog d so that the body surface temperature measurement device 1c closely contacts the body of the dog d. Furthermore, two auxiliary belts 5a extending from the mounting belt 4 are stretched around from a back side to a front side of forefeet and brought into contact with the right and left axillae of the dog d. As illustrated in FIG. 10(e), a configuration in which the auxiliary belts 5a are stretched around from the front side to the back side of the forefeet of the dog d and brought into contact with the right and left axillae of the dog d may be provided. Moreover, each of the auxiliary belts 5a includes a fastener at a tip to be connected to the buckle 50. According to the configuration described above, the auxiliary belt 5a suppresses rotation of the mounting belt 4.

The body surface temperature measurement device according to the present embodiment is also able to be expressed as follows. The body surface temperature measurement device according to the present embodiment may contact the circumference of the torso that is closer to the tail of the animal than the right and left forefeet thereof and include the auxiliary belt that makes contact across the neck on the ventral side of the animal. According to the configuration described above, it is possible to suppress deviation of a measuring position of the body surface temperature. That is, it becomes easy to measure the body surface temperature of the animal at a position desired by a user.

The body surface temperature measurement device according to the present embodiment may include the auxiliary belts that contact the right and left axillae of the animal. According to the configuration described above, it is possible to suppress deviation of a measuring position of the body surface temperature. That is, it becomes easy to measure the body surface temperature of the animal at a position desired by a user.

Moreover, the aforementioned auxiliary belts may include both a belt contacting a circumference of a torso and a belt contacting an axilla.

Furthermore, the aforementioned auxiliary belt 5a may not only assist the mounting belt 4 but also function as a device for another sensor. More specifically, another sensor may be connected to the auxiliary belt 5a. For example, another temperature sensor may be connected to the auxiliary belt 5a and used to correct a measurement value of the temperature sensing unit 2. In another example, with a configuration in which the auxiliary belt 5a includes an electrode member to make contact with the body surface of the animal and thereby allows detection of myoelectricity around the electrode member, activity of the animal may be measured. In still another example, a configuration in which a potential difference between electric potentials detected at right and left axillae of the animal are measured to thereby allow detection of an electrocardiogram may be provided.

### Embodiment 4

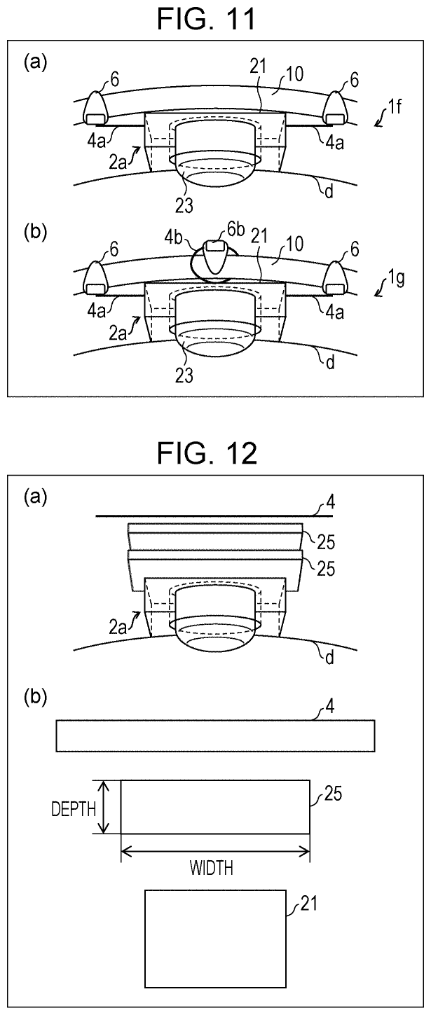

Another embodiment will be described as follows with reference to FIGS. 11 and 12. Note that, for convenience of description, a member having the same function as that of the member described in the aforementioned embodiments will be given the same reference sign and description thereof will be omitted.

In the present embodiment, each of body surface temperature measurement devices 1f and 1g is configured so as to be able to be mounted on a collar or the like that is mounted on the animal. Hereinafter, an example of mounting the body surface temperature measurement device 1f or the body surface temperature measurement device 1g on a collar 10 that is mounted on the animal will be described. Note that, the body surface temperature measurement device 1f and the body surface temperature measurement device 1g may be configured to be mounted not only on the collar 10 but also on a harness (body belt), for example. In the present embodiment, a configuration in which each of the body surface temperature measurement device 1f and the body surface temperature measurement device 1g includes the temperature sensing unit 2a will be described, but a configuration in which each of the body surface temperature measurement device 1f and the body surface temperature measurement device 1g includes the temperature sensing unit 2 instead of the temperature sensing unit 2a may be provided.

A structure of the body surface temperature measurement device 1f will be described with reference to FIG. 11. FIG. 11(a) is a view illustrating the structure of the body surface temperature measurement device 1f. As illustrated in FIG. 11(a), the body surface temperature measurement device 1f is provided with a mounting belt 4a by which the temperature sensing unit 2a is fixed to the collar 10. For detailed explanation, a connection jig 6 is provided at a tip of the mounting belt 4a. The connection jig 6 is, for example, a clip and is connected to the collar 10. The cover 23 is pressed against the body surface of the dog d and fixed by tension of the collar 10.

Another example of the body surface temperature measurement device 1g that includes a connection jig 6b will be described with reference to FIG. 11(b). FIG. 11(b) is a view illustrating a structure of the body surface temperature measurement device 1g. As illustrated in FIG. 11(b), the body surface temperature measurement device 1g includes the mounting belt 4a provided with the connection jig 6, similarly to the body surface temperature measurement device 1f described above. Furthermore, the body surface temperature measurement device 1g includes a mounting belt 4b by which the temperature sensing unit 2a (body surface temperature measurement device 1g) is fixed to the collar 10. Note that, the body belt, the harness, animal clothing, or the like may be used instead of the collar 10.

The mounting belt 4b is orthogonal to the mounting belt 4a on a back side opposite to a surface of the substrate 21 on which the auxiliary pedestal 24, the cover 23, and the like are provided. As illustrated in FIG. 11(b), in a state in which the body surface temperature measurement device 1g is mounted on the collar 10, the mounting belt 4b goes around the collar 10 to fix the body surface temperature measurement device 1g to the collar 10. Furthermore, the mounting belt 4b includes the connection jig 6b by which the body surface temperature measurement device 1g is fixed to the collar 10. A clip or the like is able to be applied as the connection jig 6b similarly to the connection jig 6. In the exemplification described above, the connection jig 6 has been described as a clip, but is not limited thereto, and a jig generally used as a fixture or a connecter, such as a buckle, a snap button, or a hook-and-loop fastener, may be used as the connection jig 6.

Moreover, by assuming that the collar 10 is mounted on the dog d relatively loosely, a configuration in which a thickness (height) of the temperature sensing unit 2a of the body surface temperature measurement device 1g or 1f is changeable may be provided. For example, a configuration in which the auxiliary pedestal 24 has a layered structure and the height of the temperature sensing unit 2a is able to be adjusted by changing the number of layers may be provided. In this configuration, an upper limit of the height of the auxiliary pedestal 24 may be set as a height of the cover 23 and a lower limit of the height of the auxiliary pedestal 24 may be set as a height obtained by subtracting a height of the curved surface of the cover 23 from the entire height of the cover 23.

Furthermore, a configuration in which a sensor pedestal (hereinafter, described as an adjuster 25) different from the auxiliary pedestal 24 is provided on a side of the substrate 21 on which the infrared ray detection sensor 22 is not arranged may be provided. The adjuster 25 will be described with reference to FIG. 12. FIGS. 12(a) and 12(b) are views each illustrating an example of a structure of the adjuster 25. As illustrated in FIG. 12(a), the adjuster 25 has a layered structure and the height of the temperature sensing unit 2a is able to be adjusted by changing the number of layers.

In the structure of the body surface temperature measurement device 1g or 1f that includes the structure 25, it is preferable that the mounting belt 4 and the substrate 21 of the temperature sensing unit 2a are configured to be separable from each other. For example, the body surface temperature measurement device 1g or 1f may have a configuration in which the adjuster 25 is inserted into a part between the mounting belt 4 and the substrate 21 and include a fixing jig (not illustrated) by which the mounting belt 4, the adjuster 25, and the substrate 21 are mutually fixed. Moreover, it is preferable that a width (length in a longitudinal direction of the mounting belt 4) of the adjuster 25 is wider than a width of the substrate 21 and a depth (length in a transverse direction of the mounting belt 4) of the adjuster 25 is wider than a depth of the mounting belt 4, as illustrated in FIG. 12(b), but there is no limitation thereto.

In the aforementioned description, the body surface temperature measurement device 1g and the body surface temperature measurement device 1f each of which includes the temperature sensing unit 2a are exemplified as illustrated in FIGS. 11 and 12, but each of the body surface temperature measurement device 1g and the body surface temperature measurement device 1f may include the temperature sensing unit 2 instead of the temperature sensing unit 2a. Moreover, a configuration in which the temperature sensing unit 2 includes the aforementioned sensor pedestal may be provided.

The body surface temperature measurement device according to the present embodiment is also able to be expressed as follows. The belt of the body surface temperature measurement device may include the connection jig for the collar or the body belt. According to the configuration described above, it becomes easier for the user to perform appropriate measurement of the body surface temperature of the animal by using the color or the body belt that he or she has.

### Embodiment 5

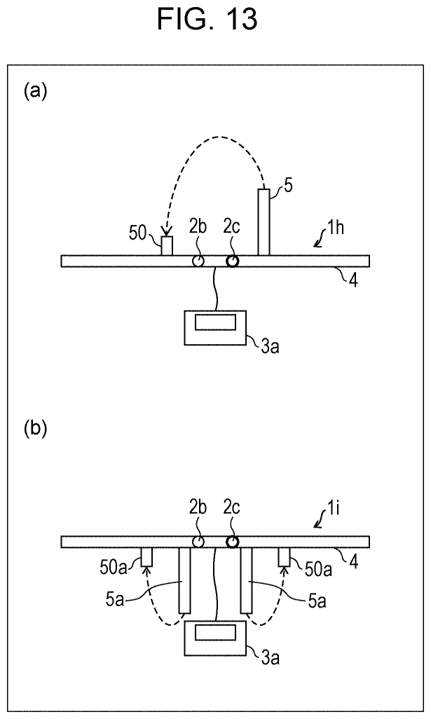

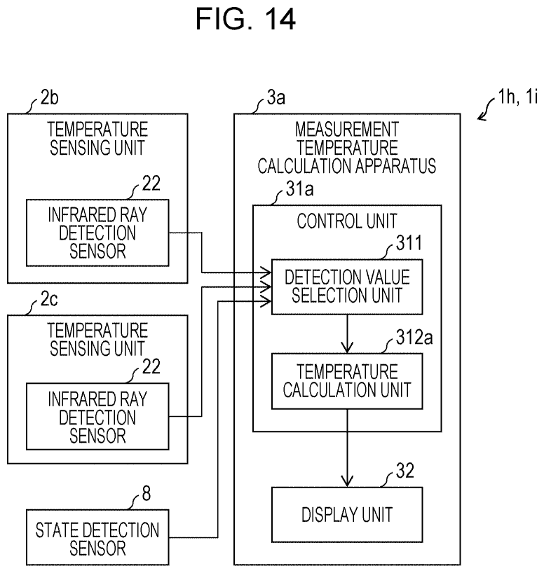

Another embodiment will be described as follows with reference to FIGS. 13 and 14. Note that, for convenience of description, a member having the same function as that of the member described in the aforementioned embodiments will be given the same reference sign and description thereof will be omitted. In the present embodiment, each of a body surface temperature measurement devices 1h and 1i includes a plurality of temperature sensing units (a temperature sensing unit 2b and a temperature sensing unit 2c).

First, structures of the body surface temperature measurement devices 1h and 1i according to an embodiment of the disclosure will be described with use of FIGS. 13 and 14. FIG. 13 is a view illustrating examples of the structures of the body surface temperature measurement devices 1h and 1i. FIG. 13(a) is a view illustrating the example of the structure of the body surface temperature measurement device 1h that has a shape of a body belt and FIG. 13(b) is a view illustrating the example of the structure of the body surface temperature measurement device 1i that has a shape of a collar.

As illustrated in FIGS. 13(a) and 13(b), each of the body surface temperature measurement devices 1h and 1i includes the plurality of temperature sensing units (the temperature sensing unit 2b and the temperature sensing unit 2c), the measurement temperature calculation apparatus 3a, the mounting belt 4, and the auxiliary belt 5 (or the auxiliary belt 5a). The mounting belt 4, the auxiliary belt 5, and the auxiliary belt 5a have been described in detail in the aforementioned embodiments, description thereof will be omitted here. Each of the temperature sensing unit 2b and the temperature sensing unit 2c has a configuration similar to that of the temperature sensing unit 2 or the temperature sensing unit 2a. Note that, in the present embodiment, an example in which a body surface temperature measurement device includes two temperature sensing units will be illustrated, but the number of temperature sensing units is not particularly limited as long as being more than one.

On the other hand, FIG. 14 is a view illustrating an example of a configuration of the measurement temperature calculation apparatus 3a. As illustrated in FIG. 14, each of the body surface temperature measurement device 1h and the body surface temperature measurement device 1i includes a state detection sensor (second sensor) 8 in addition to the configuration illustrated in FIG. 13. The state detection sensor 8 is a sensor by which a state of the body surface temperature measurement device 1h or 1i is detected, and an example thereof includes a pressure sensor, an illumination sensor, or an acceleration sensor. The state detection sensor 8 transmits a detection value to the measurement temperature calculation apparatus 3a. Note that, the state detection sensor 8 may be accommodated in a housing that is the same as that of the measurement temperature calculation apparatus 3a or a housing may be separately provided and mounted on the animal by the mounting belt 4, a belt that is separately provided, clothing, or the like, or the state detection sensor 8 may be provided in a communication terminal (such as a portable phone or a smartphone, for example) of the owner of the animal or may be provided in a place (such as a cage, a house, or a room) where the animal exists.

In the configuration illustrated in FIG. 14, the measurement temperature calculation apparatus 3a receives detection values from a plurality of temperature sensing units. On the other hand, as an example of a main part of a configuration of the measurement temperature calculation apparatus 3 that has been described in Embodiments 1 to 4 described above, a configuration in which the measurement temperature calculation apparatus 3a illustrated in FIG. 14 receives a detection value from one temperature sensing unit 2 or 2a is able to be cited.

Next, the measurement temperature calculation apparatus 3a will be described. The measurement temperature calculation apparatus 3a includes a control unit 31a and the display unit 32. The control unit 31a includes the detection value selection unit 311 and the temperature calculation unit 312a.

The detection value selection unit 311 selects, in accordance with detection values of the plurality of the temperature sensing units and a detection value of the state detection sensor 8, a detection value by which measurement temperature is calculated from the detection values of the plurality of temperature sensing units.

For detailed explanation, the detection value selection unit 311 receives detection values that are detected by the temperature sensing unit 2b and the temperature sensing unit 2c. Moreover, in a case where a change per predetermined time of the respective temperature sensing units is within a predetermined value, the detection value selection unit 311 selects detection values of the temperature sensing units as detection values that are used to calculate the measurement temperature and transmits the detection values to the temperature calculation unit 312a. In other words, the detection value selection unit 311 does not select a detection value of a temperature sensing unit having an extreme change as a detection value by which the measurement temperature is calculated. Note that, in the configuration described above, the state detection sensor 8 is not an essential component.

Moreover, the detection value selection unit 311 selects a detection value of a temperature sensing unit, which is used to calculate the measurement temperature, in accordance with the detection value of the state detection sensor 8. For example, in a configuration in which the state detection sensor 8 is a pressure sensor, the detection value selection unit 311 selects a detection value of a temperature sensing unit on a side where pressure is not applied. Specifically, the detection value selection unit 311 determines, from the detection value of the state detection sensor 8, a side where the dog is not covered with something or the dog is not pressed by something (a temperature sensing unit on the side). Then, as the detection value that is used to calculate the measurement temperature, the detection value selection unit 311 selects a detection value of a temperature sensing unit on the side where nothing covers or on the side where nothing presses. In a configuration in which the state detection sensor 8 is an illumination sensor, the detection value selection unit 311 determines, from a detection value of the illumination sensor, whether or not something covers a temperature sensing unit. Then, as the detection value that is used to calculate the measurement temperature, the detection value selection unit 311 selects a detection value of a temperature sensing unit on the side where nothing covers, or the detection value selection unit 311 determines a side that is directly exposed to sunlight from a detection value of illumination which exceeds a predetermined threshold. The detection value selection unit 311 selects, as the detection value that is used to calculate the measurement temperature, a detection value of a temperature sensing unit on a side that is not directly exposed to sunlight. In a configuration in which the state detection sensor 8 is an acceleration sensor, the detection value selection unit 311 specifies a direction of the dig d from a detection value of the acceleration sensor. Then, the detection value selection unit 311 selects, as the detection value that is used to calculate the measurement temperature, a detection value of a temperature sensing unit that is not positioned on a lower side (where there is a possibility that the sensor is covered with the dog itself) of the dog d.

The temperature calculation unit 312a calculates a measurement temperature from at least one detection value of the detection values detected by the plurality temperature sensing units. Specifically, the measurement temperature calculation apparatus 3a calculates the measurement temperature from at least one detection value among the detection values of the plurality of temperature sensing units (the temperature sensing unit 2b and the temperature sensing unit 2c). For example, the measurement temperature calculation apparatus 3a calculates temperatures by the detection values that are received from the plurality of temperature sensing units and sets an average value of the calculated temperatures to a measurement temperature. The temperature calculation unit 312a transmits the measurement temperature that is calculated to the display unit 32. The display unit 32 displays the measurement temperature, an operation state of the measurement temperature calculation apparatus 3a, or the like.

Note that, in the aforementioned description of the present embodiment, the body surface temperature measurement device that includes the auxiliary belt 5 or the auxiliary belt 5a has been described, but the configuration of the present embodiment may be applied to the body surface temperature measurement device that does not include the auxiliary belt 5 or the auxiliary belt 5a, which is described in Embodiment 1.

It is preferable that arrangement positions of the temperature sensing units 2b and 2c are positions where there is no interference from outside so that the temperature sensing units 2b and 2c obtain correct detection values. The interference from outside means a physical pressure with respect to the temperature sensing unit 2b and 2c, for example, such as a pressure caused when the animal changes a posture and thereby contacts something such as the ground, a floor surface, a wall surface, or a futon, or a physical pressure received from movement of a skeleton of the animal itself. For example, a dog is less likely to lie so as to face directly upward. Thus, by arranging the temperature sensing units 2b and 2c right over the spine, it is possible to reduce the interference from outside. On the other hand, in the case of arranging the temperature sensing units 2b and 2c right over the spine, the temperature sensing units 2b and 2c interfere with the spine. Accordingly, in a case of a dog of a medium size or more, it is preferable that the temperature sensing units 2b and 2c are arranged on an outside (left side or right side) of the spine and in a region inside a scapula. In the region, a forefoot or a hind foot of the dog do not reach, and there is little interference from outside.

In a case of a small size dog, a region between a spine and a scapula is not large enough. Thus, in a case of arranging the temperature sensing units 2b and 2c in the region, the spine or scapula collides with the temperature sensing unit 2b or 2c, thus giving stress to the dog. Accordingly, in the case of the small size dog, it is necessary to arrange the temperature sensing units 2b and 2c on an outside of the scapula.

According to the configuration of the body surface temperature measurement device 1h or 1i described in the present embodiment, the detection value selection unit 311 selects, in accordance with the detection values of the plurality of the temperature sensing units or the detection value of the state detection sensor 8, the detection value that is used to calculate the measurement temperature among the detection values of the plurality of temperature sensing units. Thus, the detection value selection unit 311 is able to use, from the detection values of the plurality of the temperature sensing units or the detection value of the state detection sensor 8, the detection value of the temperature sensing unit that is determined not to receive the interference and calculate the measurement temperature. Accordingly, the configuration of the body surface temperature measurement device 1h or 1i is particularly effective in a case where the temperature sensing units 2b and 2c are arranged at positions where there is a high possibility that the interference from outside is received.

The body surface temperature measurement device according to the present embodiment is also able to be expressed as follows. The body surface temperature measurement device includes the plurality of temperature sensing units including the temperature sensing unit and the measurement temperature calculation apparatus that calculates the measurement temperature from at least one detection value of the detection values detected by the plurality of temperature sensing units, and the measurement temperature calculation apparatus calculates the measurement temperature from the detection values of the plurality of temperature sensing units. Thus, body surface temperature measurement with high accuracy is easily performed.

Moreover, the body surface temperature measurement device may include the plurality of temperature sensing units including the temperature sensing unit, the measurement temperature calculation apparatus that calculates the measurement temperature from at least one detection value of the detection values detected by the plurality of temperature sensing units, and the second sensor that detects a state of an animal, in which the measurement temperature calculation apparatus may include the detection value selection unit that selects, in accordance with the detection values of the plurality of temperature sensing unit or the detection value of the second sensor, the detection value that is used to calculate the measurement value from the detection values of the plurality of temperature sensing units.

According to the aforementioned configuration, the detection value that is used to calculate the measurement temperature is selected from the detection values of the plurality of temperature sensing units in accordance with the detection values of the plurality of temperature sensing units. For example, a configuration in which, under consideration of progress of the detection values of the temperature sensing units, a detection value of a temperature sensing unit having an extreme change is not used to calculate the measurement temperature may be provided. Thus, body surface temperature measurement according to a state of the animal is easily performed.

Moreover, according to the aforementioned configuration, the detection value that is used to calculate the measurement temperature is selected from the detection values of the plurality of temperature sensing units in accordance with the detection values of the plurality of temperature sensing units or the detection value of the second sensor. For example, in a case where the second sensor is a pressure sensor, a configuration in which a detection value of a temperature sensor arranged on a side of the animal where pressure is not applied is used to calculate the measurement temperature may be provided. In a case where the second sensor is an illumination sensor, a configuration in which whether or not something (for example, such as clothing) covers a temperature sensor is determined from a detection value of the illumination sensor and a detection value of a temperature sensor that is covered with nothing is used to calculate the measurement temperature may be provided. In a case where the second sensor is an acceleration sensor, a configuration in which a posture of the animal is determined and the measurement temperature is calculated from a detection value of a temperature sensor that is not covered with the animal (that is not buried under the animal) may be provided. Thus, body surface temperature measurement according to a state of the animal is easily performed.

The disclosure is not limited to each of the embodiments described above and may be modified in various manners within the scope of the claims and an embodiment achieved by appropriately combining technical means disclosed in each of different embodiments is also encompassed in the technical scope of the disclosure. Further, by combining the technical means disclosed in each of the embodiments, a new technical feature may be formed.

This application claims priority based on Japanese Patent Application No. 2016-235703 filed in Japan on Dec. 5, 2016, the entire content of Japanese Patent Application No. 2016-235703 is incorporated this application claims by reference.

### REFERENCE SIGNS LIST

1, 1b, 1c, 1f to 1i body surface temperature measurement device
2, 2a to 2c temperature sensing unit
22 infrared ray detection sensor
23 cover
24 auxiliary pedestal
3, 3a measurement temperature calculation apparatus
311 detection value selection unit
4, 4a, 4b mounting belt (belt)
5, 5a auxiliary belt
6, 6b connection jig

1: A body surface temperature measurement device that measures a body surface temperature of an animal, the body surface temperature measurement device comprising:
a temperature sensing unit that includes an infrared ray detection sensor and a cover that covers the infrared ray detection sensor; and
a belt that causes the temperature sensing unit to closely contact a body surface of the animal or that fixes the temperature sensing unit to a collar to be mounted on the animal or a body belt to be mounted on the animal; wherein
in a case where the body surface temperature measurement device is mounted on the animal, the cover is pressed against the body surface of the animal, and
at least a part of a contact surface of the cover, which contacts the body surface of the animal, is formed into a curved surface that bulges toward a body surface side of the animal.

2: The body surface temperature measurement device according to claim 1, further comprising a measurement temperature calculation apparatus that calculates a measurement temperature from a detection value that is detected by the temperature sensing unit.

3: The body surface temperature measurement device according to claim 1, wherein the temperature sensing unit further includes an auxiliary pedestal that covers at least a part of the cover.

4: The body surface temperature measurement device according to claim 3, wherein the auxiliary pedestal is made from an elastic member.

5: The body surface temperature measurement device according to claim 1, wherein the belt includes an auxiliary belt by which a position of the belt on the body surface of the animal is fixed.

6: The body surface temperature measurement device according to claim 1, wherein the belt includes a connection jig for the collar or the body belt.

7: The body surface temperature measurement device according to claim 3, wherein
a hole is provided in the auxiliary pedestal, and
the cover is inserted into the hole.

8: The body surface temperature measurement device according to claim 1, further comprising:
a plurality of temperature sensing units that include the temperature sensing unit;
a measurement temperature calculation apparatus that calculates a measurement temperature from at least one detection value of detection values detected by the plurality of temperature sensing units; and
a second sensor that detects a state of the animal, wherein
the measurement temperature calculation apparatus includes a detection value selection unit that selects, in accordance with the detection values of the plurality of temperature sensing units or a detection value of the second sensor, the detection value that is used to calculate the measurement temperature from the detection values of the plurality of temperature sensing units.
